(19) United States
(12) Patent Application Publication
(10) Pub. No.: US 2017/0056126 A1
(43) Pub. Date: Mar. 2, 2017

(54) WRONG SITE SAFE SURGERY

(71) Applicant: Black Cat Medical LLC, Fortville, IN (US)

(72) Inventor: Michael F. Guzman, Fortville, IN (US)

(21) Appl. No.: 14/933,834

(22) Filed: Nov. 5, 2015

Related U.S. Application Data
(60) Provisional application No. 62/208,951, filed on Aug. 24, 2015.

(51) Int. Cl. A61B 90/90; A61B 90/96; B41J 3/407; G09F 3/10

(52) U.S. Cl. CPC A61B 90/90; G09F 3/10; A61B 90/96; B41J 3/4073

(57) ABSTRACT

A labeling system for use on a human surgical patient is disclosed. The labeling system includes a label with a main sheet that has a top and bottom surface. An adhesive that is suitable for adhering to the skin of a patient may be present on the bottom surface of the main sheet. Readable indicia may be visible on the top surface or through the top surface of the main sheet. The readable indicia may include affirmative indicia indicating a location for surgery and/or negative indicia indicating a location not for surgery.

### CROSS REFERENCE TO RELATED APPLICATIONS

This application claims the benefit of U.S. Provisional Application No. 62/208,951 filed Aug. 24, 2015, which is hereby incorporated by reference in its entirety.

### BACKGROUND

The present invention relates to surgery, and more specifically to labels for reducing the chance of surgery at the wrong site of a given patient.

Although there are procedures in place to avoid performing operations or surgeries on the wrong part of the body, wrong site surgeries continue to occur. A surgeon may operate on the incorrect side of the body, for instance, performing a procedure on the left eye when the procedure should have been performed on the right eye. In some cases, the operation may be performed on the correct body part but in the incorrect location on that body part. In other cases, the surgeon may operate on the correct body part but may perform the incorrect procedure. Wrong site surgery can have very serious medical consequences for patients on whom the surgery is performed. Therefore, it would be desirable to provide a system that could reduce the occurrence of these mistakes.

Thus, there is a need for improvement in this field.

### SUMMARY

The claims, and only the claims, recite the invention. In summary, it may include a labeling system for use on a human surgical patient. The labeling system includes a label that comprises a main sheet having a top surface and a bottom surface. The bottom surface may include an adhesive that is suitable for adhering to the skin of the patient. A removable backing may cover the adhesive prior to use. Readable indicia on the main sheet are visible on or through said top surface. The readable indicia may include affirmative indicia indicating a location for surgery and/or negative indicia indicating a location not for surgery.

These and other features are provided.

### BRIEF DESCRIPTION OF THE DRAWINGS

FIG. 1 is a top perspective view of a label.

FIG. 2 is a top plan view of a first example of a label, showing negative indicia on or through a top surface of a sheet.

FIG. 3 is a top plan view an example of a label, showing affirmative indicia on or through a top surface of a sheet.

FIG. 4 is a top plan view of an example of a label, showing both affirmative and negative indicia on or through a top surface of a sheet.

FIG. 5 is a top plan view of an example of a label, showing confirmation indicia on or through a top surface of a sheet.

FIG. 6 is a top plan view of an example of a label, showing negative indicia and identification indicia on or through a top surface of a sheet.

FIG. 7 is a top plan view of an example of a label, showing negative indicia and identification indicia on or through a top surface of a sheet.

FIG. 8 is a top plan view of an example of a label, showing negative indicia on or through a top surface of a sheet.

FIG. 9 is a top plan view of an example of a label, showing negative indicia and identification indicia on or through a top surface of a sheet.

FIG. 10 is a top plan view of an example of a label, showing affirmative and negative indicia and identification indicia on or through a top surface of a sheet.

FIG. 11A is a top plan view of an example of a label, showing both affirmative and negative indicia on or through a top surface of a sheet.

FIG. 11B is a bottom plan view of an example of a label, such as the type shown in FIG. 11A, showing one example of tattoo ink on and in the adhesive.

FIG. 11C is cross-sectional view taken along lines 11C-11C in FIG. 11A, showing one example of tattoo ink on and in the adhesive.

FIG. 12 is a top plan view of an example of a label, showing confirmation indicia and identification indicia on or through a top surface of a sheet.

FIG. 13 is a schematic representation of a system for printing labels for a medical procedure from a template.

FIG. 14 is a flowchart for one method for indicating on a human surgical patient.

### DESCRIPTION OF THE SELECTED EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. One embodiment of the invention is shown in great detail; although it will be apparent to those skilled in the relevant art that some features that are not relevant to the present invention may not be shown for the sake of clarity.

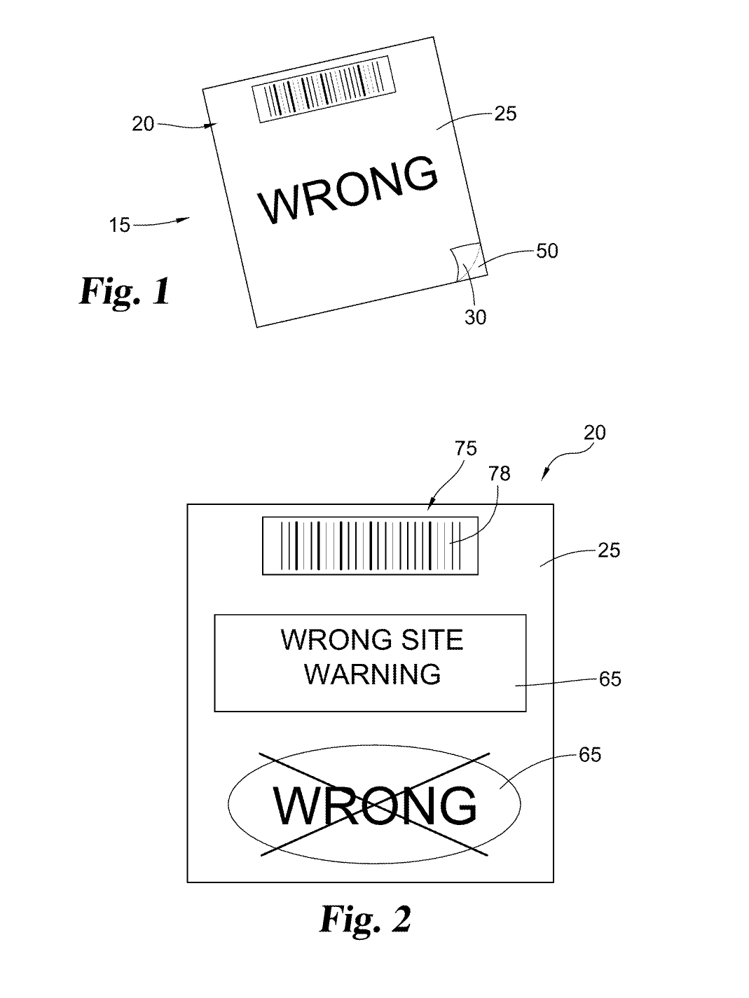

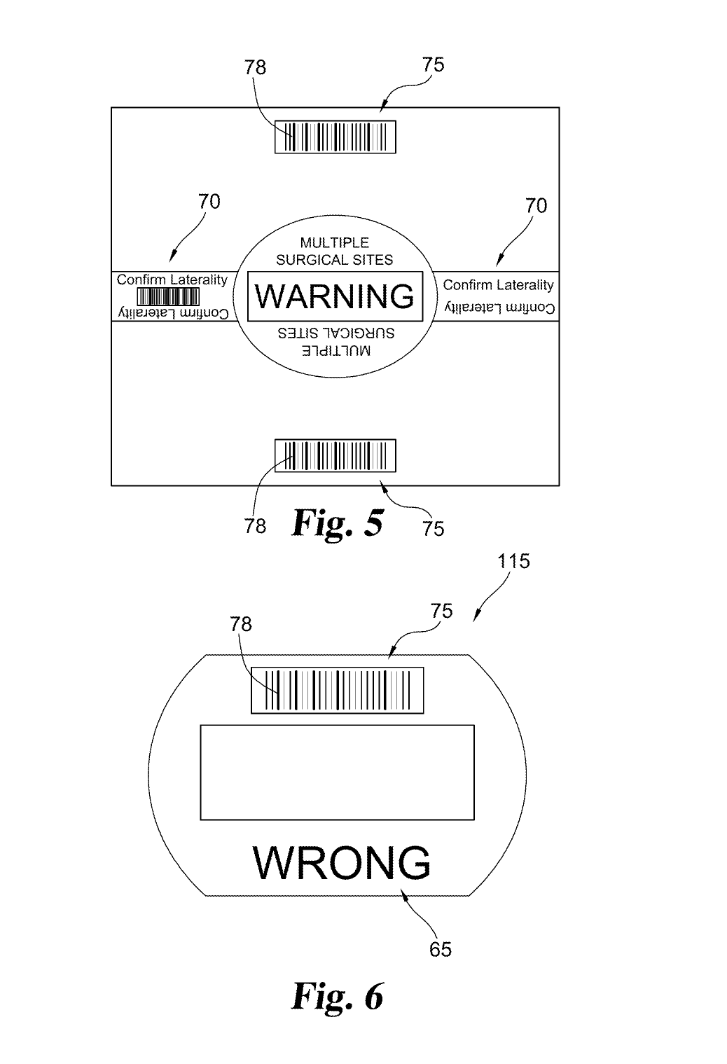

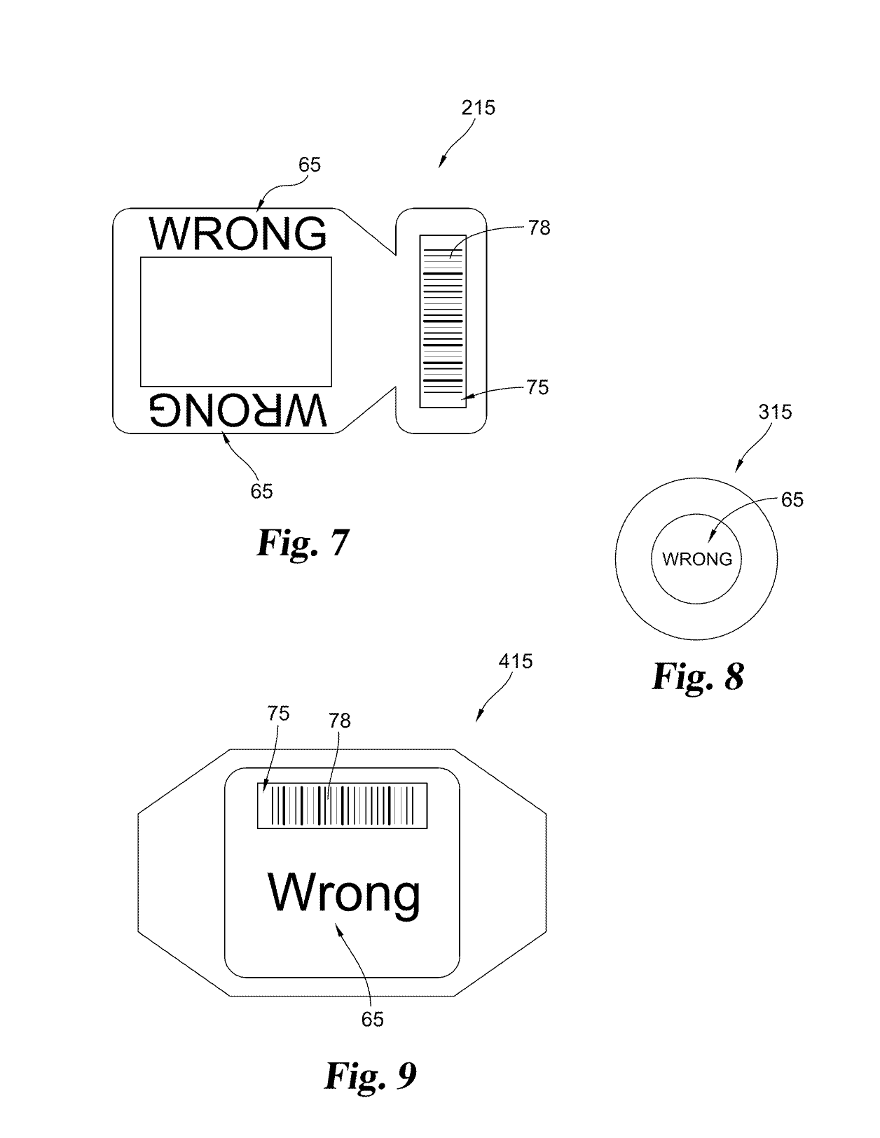

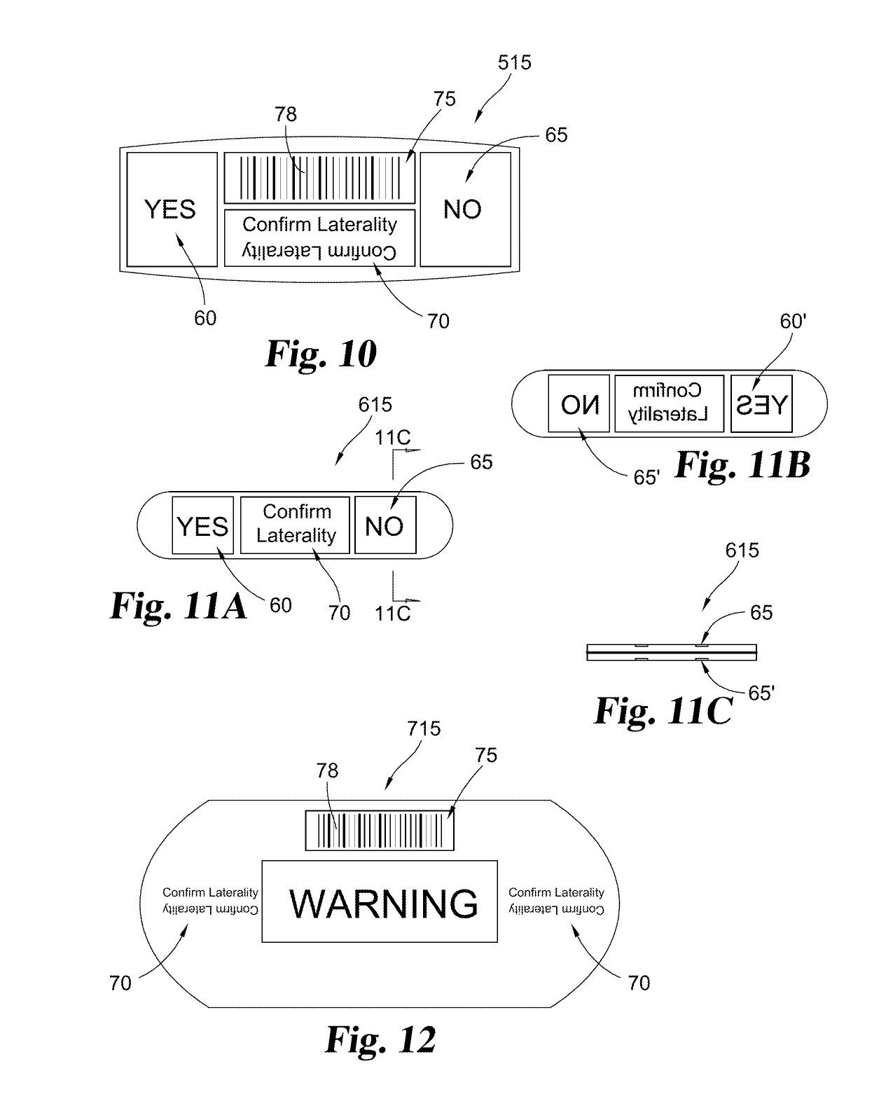

With reference to the drawing Figures, for example FIGS. 1-13, a labeling system for use on a human surgical patient is shown. A label 15 may include a main sheet 20 having a top surface 25 and a bottom surface 30. An adhesive suitable for adhering to human skin is provided on most or all of the bottom surface 30 of the main sheet 20. Preferably, the adhesive is covered by a removable backing 50, such as a peel-off backing, that is to be removed by the healthcare provider prior to applying the label 15 to the skin of a patient. The main sheet 20 includes readable indicia that are visible on or through the top surface. The readable indicia may include affirmative indicia 60 indicating a location for surgery and/or negative indicia 65 indicating a location not for surgery.

One feature that is optional, but not required, may be that the affirmative indicia 60 and the negative indicia 65 are differentiated from each other so that one corresponds to medial anatomy and the other corresponds to lateral anatomy (see FIG. 4).

One feature that is optional, but not required, may be that the affirmative indicia 60 and the negative indicia 65 are differentiated from each other so that one corresponds to left anatomy and the other corresponds to right anatomy.

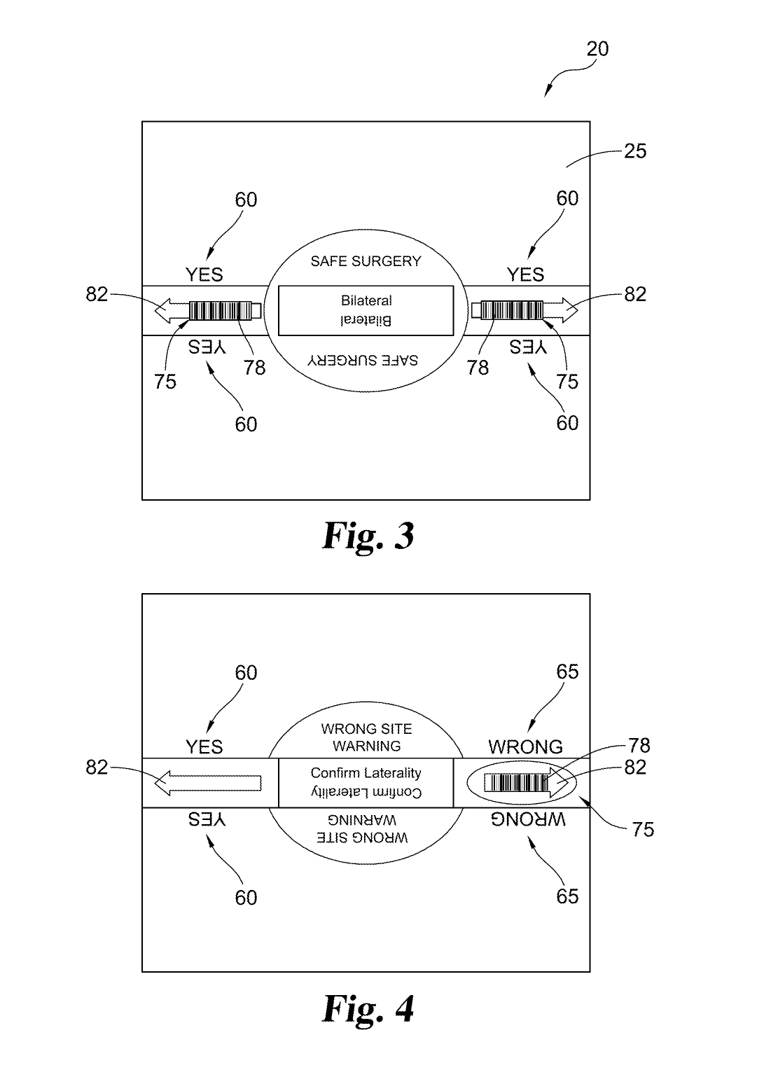

One feature that is optional, but not required, may be that the affirmative indicia 60 is selected from a group of words or symbols including one or more of: “yes”, “cut”, “surgery”, “correct”, “right”, “affirmative”, the color green (see FIG. 3).

One feature that is optional, but not required, may be that the negative indicia 65 is selected from a group of words or symbols including one or more of: “no”, “no cut”, “no surgery”, “not correct”, “wrong”, “negative”, the color red (see FIG. 2).

One feature that is optional, but not required, may be that the readable indicia may include confirmation indicia 70, such as the term “confirm laterality”, to encourage a surgeon, nurse, or other medical staff to ensure that surgery is performed at the correct site (see FIG. 3,4, or 5).

One feature that is optional, but not required, may be that any of the readable indicia, such as affirmative indicia 60, negative indicia 65, or confirmation indicia 70 may be printed on the label 15 in multiple directions to ensure readability from different vantage points.

One feature that is optional, but not required, may be that the readable indicia includes identification indicia 75 on said main sheet 20 and visible on or through said top surface. The identification indicia 75 may include information corresponding to the patient's identity, the planned surgery for the patient, or both. These identification indicia 75 may be handwritten or printed wording, or may include at least one machine readable identifier 78, such as a bar code, that corresponds to the patient's identity, the planned surgery for the patient, or both.

One feature that is optional, but not required, may be that the readable indicia may include at least one arrow 82. The arrow 82 may point to correct or incorrect sites for surgery and may include additional affirmative 60 or negative indicia 65 that indicates where surgery should take place and where surgery should not take place. Machine readable identifiers 78 that correspond to the patient's identity and/or the planned surgical procedure may be included on the arrows 82.

One feature that is optional, but not required, may be that the main sheet 20 may be more than one layer. For example, the main sheet 20 may include a layer of printable material (plastic, paper, a combination, or otherwise) with an optional clear or translucent covering thereover.

One feature that is optional, but not required, may be that the labels 15 are available in a variety of different shapes and sizes to allow use with different areas of the body or for use in pediatrics, and head/neck procedures.

One feature that is optional, but not required, may be that instead of or in addition to indicia regarding medial vs. lateral positioning, and/or left vs. right surgery, the labels 15 may also provide indicia regarding caudal vs. cranial positioning and/or regarding ventral vs dorsal positioning.

One feature that is optional, but not required, may be that the labels 15 may be formed from different varieties of bandages or dressings (see FIGS. 6-12). Some of these labels 115, 215, 315, 415 may include only negative indicia 65. Other labels 515, 615, 715 may include both affirmative indicia 60 and negative indicia 65, as well as confirmation indicia 70 for application to sites that are to be operated on. In additional embodiments, any other desired shape or type of bandages and/or dressings may be used as a label. Any desired shape or type of bandage may include any desired combination of affirmative indicia 60, negative indicia 65, confirmation indicia 70, or identification indicia 75.

Also optionally, second indicia may be printed or otherwise provided within the adhesive material, preferably on the bottom-most portion of the adhesive, using a temporary ink that may adhere to the skin of a patient. Such temporary ink shall hereafter be referred to as “tattoo ink” or “tattoo indicia”. Some of the tattoo ink may bleed or transfer onto the patient's skin in the form of some or all of the readable indicia. The tattoo ink may be printed in reverse mirror image. For example, such a tattoo ink may be printed in reverse mirror image of, for example, “NO” or other negative indicia 65 such that when the readable indicia transfers onto the skin when label 15 is adhered to a patient. FIGS. 11A-C show an example, with indicia 65 on or in the main body of label 615, and a mirror image of that indicia as tattoo ink 65′ on or in the adhesive layer on the bottom (see FIGS. 11B and C). Such tattoo ink may be the reverse mirror image (compare FIG. 11A vs. 11B), of some or all of the indicia on the main sheet, although other arrangements are possible as well. For example, only a subset of top indicia 65 may be as tattoo indicia 65′, and/or they may or may not be in registry with each other, and/or of different size, fonts, etc. Moreover, in addition to or in lieu of top indicia 65, instead the main sheet and/or adhesive of label 615 may be clear, transparent or translucent, allowing indicia 65′ to be viewable (and machine and/or human readable) through the main sheet. These features are not limited to the arrangement of label 615 in FIGS. 11A-C, but rather may be used in whole or in part in the other labels draw, described and/or claimed herein.

The tattoo ink provides the optional feature that in the event that the label 15 becomes separated or otherwise falls off of the patient, there is still a residual ink mark left from the tattoo ink on the skin through surgery, directing the healthcare provider as to where the surgical site is and is not. Optionally, the tattoo ink may be most or the only indicia, with the main sheet 20 being transparent or translucent, allowing visualization of the indicia through main sheet 20.

Optionally, the labels 15 may be preprinted kept in inventory. Such labels 15 might optionally include preprinted affirmative indicia 60, negative indicia 65, confirmation indicia 70, and/or identification indicia 75. The identification indicia 75 may be used to indicate the type of surgery for which the set of labels is to be used.

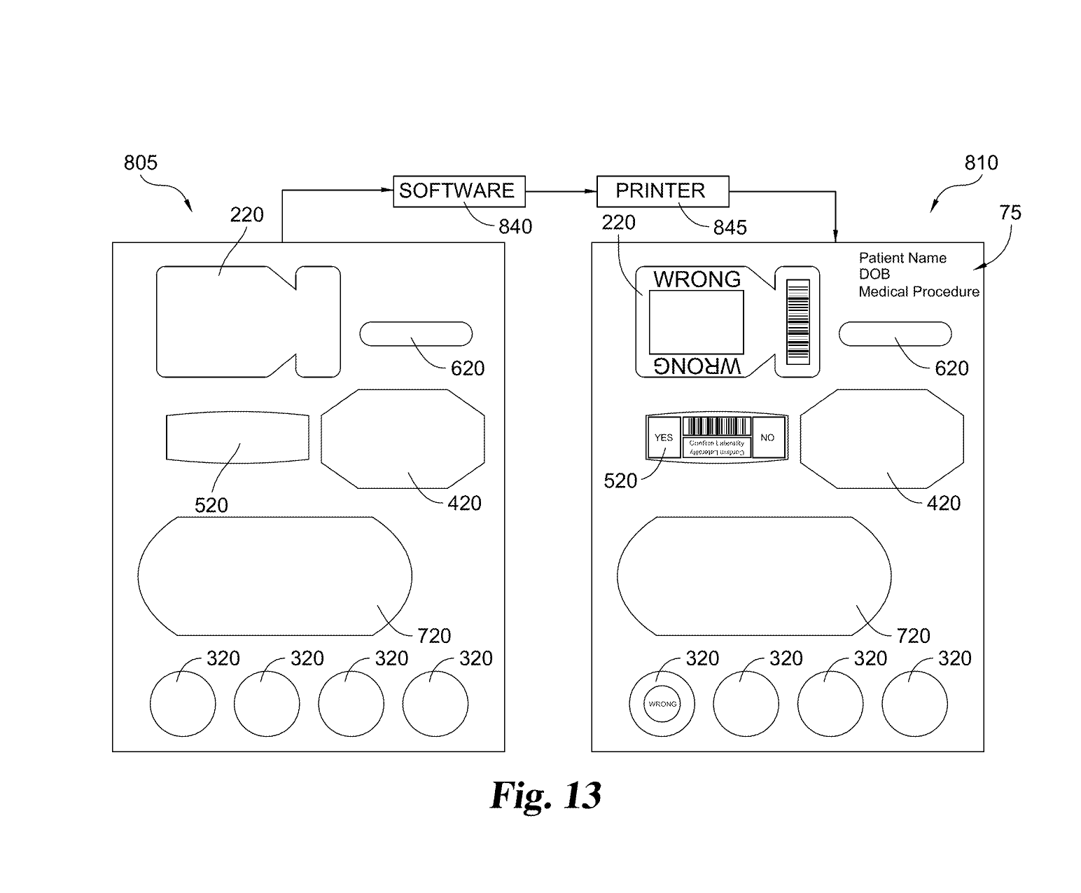

Another option is that the labels 15 may be fully or partially custom printed for a specific type of surgery. As shown in FIG. 13, a blank sheet of labels 805 may be provided with various shapes and sizes of labels. Custom software 840 is accessed that contains premade label templates for various medical procedures. The software may allow a user to manually select the label template for the medical procedure to be performed or medical information may be automatically accessed from a medical records database to determine the correct label template. The printer prints corresponding labels 810 for the corresponding surgeries based according to the selected template. The needed labels for the given medical procedure are printed and labels that are not necessary may be left blank. Additionally, additional information such as the patient's name, patient's date of birth, bar code identification information corresponding to the patient, or any other suitable data may be printed on the sheet of labels 810.

Such printing optionally, but not necessarily, may include providing a label according to any one or more labels disclosed, drawn or claimed herein, by first providing label sheet 805 that is at least partially unprinted. One may store in a computer database information about the patient including: (i) patient identifying information including at least the patient's name; and, (ii) at least one prescribed surgery for the patient to receive, and also store separate database records about several various types of surgeries, including in such records: (i) surgery identifier; and, (ii) information to associate that surgery identifier with information to be printed on the surgery label. The act of associating in a computer database the prescribed surgery for the patient to receive and the information to be printed on the surgery label is then followed by custom printing in the label sheet that is at least partially unprinted the information to be printed on the surgery label to create the label according to any one or more labels disclosed, drawn or claimed herein which custom corresponds to the patient and the patient's prescribed surgery. As but one example, unprinted sheet 805 in FIG. 3 is printed to create label 810.

Optionally, but not necessarily, may include label sheet 805 that is at least partially unprinted comprises at least two separate pre-cut-out portions (shown on the left side of FIG. 13 for mere examples as 220, 320, 420, 520, 620 and/or 720), on a common label; and wherein the information to be printed on at least a first of the pre-cut-out portions both (shown on the right side of FIG. 13 for mere examples as 220, 520 and 320, (but optionally with the other pre-cut-out portions not custom printed): (a) is according to the finished label; and, (b) is templated from the computer database to print, during the act of printing, at a location on the label sheet on the first of the pre-cut-out portions. Or instead, there may be no, or only limited or single pre-cut-out portions. Thus, a single dedicated sheet per label may be used instead.

Optionally, but not necessarily, the methods may include printing includes printing patient identifying information 75, including in human readable form at least the patient's name, on the label sheet. Other information, date of birth, social security number, the prescribed medical procedure, and otherwise may also optionally be printed, on the pre-cut-out-portion(s), on the main sheet but outside the pre-cut-out portions, or both, or neither.

Optionally, but not necessarily, one or more database(s), (stand alone, networked, or otherwise) may include the following structure. They may include a patient data tables, with a record unique to a patient. Such data tables may include fields including the patient's name, date of birth, unique identifying alphanumeric, and/or otherwise. The patent data table may include one or more surgery identifier fields. Such fields may identify (by name, code or otherwise) the particular surgery, and/or may be linked or otherwise associated with another data table for particular surgeries (either for that patient, for that hospital, or otherwise). Such codes may be Current Procedural Terminology (CPT), federal HCPCAS codes, billing codes (U.S. or foreign) or otherwise. Moreover, that data table, but preferable a separate data table, may include information regarding a pre-existing listing of records of types of surgeries. This may include a field for type of surgery, and/or a code (see above) for that surgery, and optionally a printing template type corresponding to the template (see for example 805 and 810 in FIG. 13) for that surgery. It may include other more specific sub-indicators corresponding to whether that surgery is left or right, medial or lateral, dorsal or antior, caudal or cranial, or otherwise. Optionally, however, the database may be structured with the printing template information in a separate printing-template table (which could be altered or expanded from time to time) to correspond with various printing sheet templates. Typically, one or more fields among the tables is associated or related, or can be so upon querying, as is known generally in the field of relational database programming.

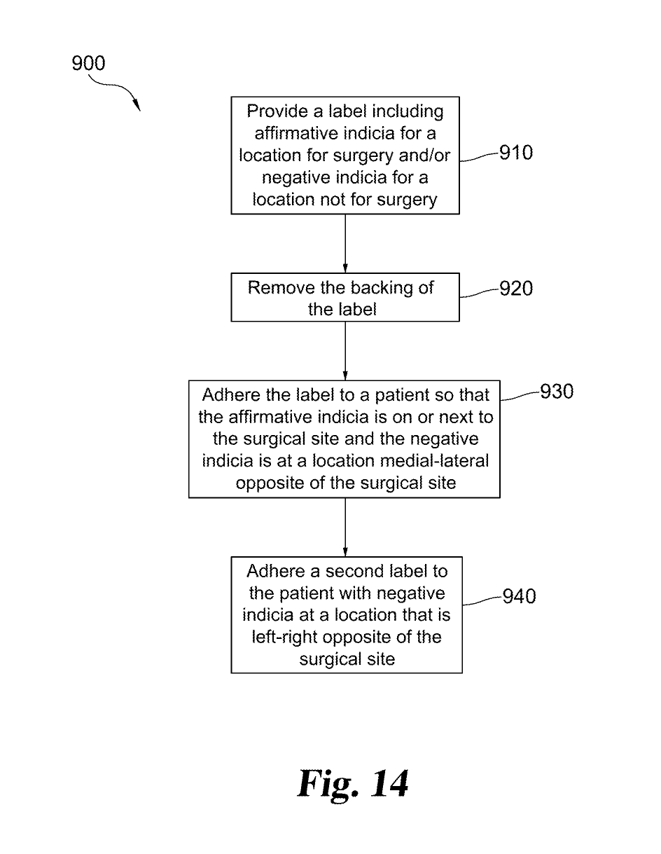

One optional method for indicating on a human surgical patient is shown in the flowchart 900 of FIG. 14. The method comprises providing 910 a label having a main sheet. The main sheet includes a top surface and a bottom surface. An adhesive suitable for adhering to the skin of a patient is present on the bottom surface. A removable backing covers the adhesive prior to use of the label. Readable indicia may be visible on or through the top surface. The readable indicia include affirmative indicia indicating a location for surgery and/or negative indicia indicating a location not for surgery. Other methods may be used as well.

The backing of the label is removed 920. Then, the label is adhered 930 to the patient so that the affirmative indicia is positioned on or next to the surgical site, and so that the negative indicia is at a location which is the medial-lateral opposite of the surgical site. As an optional feature, a second label with negative indicia may be adhered 940 to the patient at a location which is the left-right opposite of the surgical site.

One feature that is optional, but not required, may be that the label may be adhered to the patient by a nurse or other health care provided with the participation of the patient, family, guardian, or friend to ensure that the label is placed in the correct location on the body.

One feature that is optional, but not required, may be that the affirmative indicia is selected from a group of words or symbols including one or more of: “yes”, “cut”, “surgery”, “correct”, “right”, “affirmative”, the color green.

One feature that is optional, but not required, may be that the negative indicia is selected from a group of words or symbols including one or more of: “no”, “no cut”, “no surgery”, “not correct”, “wrong”, “wrong site”, “negative”, the color red.

The method may optionally include the act of reading readable indicia that includes identifying information corresponding to the patient's identity, the planned surgery, or both. Optionally, the readable indicia may be read by scanning at least one machine readable identifier, such as a bar code. The readable indicia and/or the fact that the readable indicia has been read may be subsequently recorded in a written file, a computer database, or in any other suitable format. The identification indicia may match with another form of identification such as a patient's admission bracelet to ensure that the label has been adhered to the correct location. The identification indicia may be read at different times during the medical procedure to ensure correct application of the labels. For example, the identification indicia may be read and recorded before adhering the label to the patient, immediately after adhering the label to the patient, and during a time out in the operating room.

One feature that is optional, but not required, may be that adhesion of the label to the patient is performed directly before or directly after marking the patient as required by the Joint Commission on the Accreditation of Healthcare Organizations (JCAHO).

The method may optionally include the act of accessing software that contains medical data for a patient and label templates with positions for positive and/or negative indicia on a label or a set of labels. Each of the label templates corresponds to a particular medical procedure. The corresponding template for a medical procedure that is to be performed on a patient is chosen according to the medical data. A label or a set of labels for the medical procedure to be performed is printed. The position of the positive indicia and the negative indicia on the label or set of labels is determined by the label template that corresponds to the medical procedure to be performed.

As used here (claims, specification, and other definitions) the following terms have the following meaning:

Articles and phases such as, “the”, “a”, “an”, “at least one”, and “a first”, “comprising”, “having” and “including” here are not limited to mean only one, but rather are inclusive and open ended to also include, optionally, two or more of such elements and/or other elements. In terms of the meaning of words or terms or phrases herein, literal differences therein are not superfluous and have different meaning, and are not to be synonymous with words or terms or phrases in the same or other claims.

The term “means for” in a claim invokes 35 U.S.C. §112(f), literally encompassing the recited function and corresponding structure and equivalents thereto. Its absence does not, unless there otherwise is insufficient structure recited for that claim element. Nothing herein or elsewhere restricts the doctrine of equivalents available to the patentee.

The term “and/or” is inclusive here, meaning “and” as well as “or”. For example, “P and/or Q” encompasses, P, Q, and P with Q; and, such “P and/or Q” may include other elements as well.

The term “adhesive” as used herein has the meaning a substance that is able to stick to another object. More specifically, an adhesive is a substance that may be used to stick to other objects or materials together.

The term “affirmative indicia” as used herein has the meaning any readable indicia that indicate that a certain body part or a portion of a certain body part is to be subject to a medical procedure. As an example, affirmative indicia may include the words “yes”, “cut”, “surgery”, “correct”, “right”, “affirmative”, or could be the color green without using text. Additionally, any other predetermined word, color, or symbol that indicates a site is not to be subject to a medical procedure may be used as affirmative indicia.

The term “bottom surface” as used herein has the meaning an exterior layer found on the lower most side of the main sheet and located on the side opposite the top surface.

The term “confirmation indicia” as used herein has the meaning, any readable indicia that alerts, warns, or advises an individual to check or ascertain the portion of the body which is to be subject to a medical procedure. Examples of confirmation indicia may include the terms “warning”, “confirm laterality”, “multiple surgical sites”, “bilateral”, or could be the color yellow or orange without using text.

The term “identification indicia” as used herein has the meaning, any readable indicia that indicates who someone is, indicates facts about that person, or indicates a medical procedure that is to be performed. Identifying information may be a person's name, a birthday, a phone number, or an account or patient number. Identifying information may also include medical data specific to that person such as procedures to be performed on a certain patient.

The term “label” as used herein has the meaning an object that identifies and/or gives information about another object. A label may be attached directly to the object that it is providing information, located adjacent to the object for which it is providing information, or positioned at a remote location.

The term “label template” as used herein has the meaning preset positions for affirmative indicia and negative indicia for a label or a set of labels based on a particular type of medical procedure that is to be performed. The label template may include information regarding the position of the affirmative and negative indicia in addition to information regarding the number and the types of labels that must be generated for a particular procedure.

The term “lateral” as used herein has the meaning toward the side the body or a body part. As an example, the lateral portion of the knee refers to the outside portion of the knee.

The term “left anatomy” as used herein has the meaning the left side of the body. As an example, a left arm and a left leg are part of the left anatomy.

The term “left-right opposite” as used herein has the meaning the opposing side in terms of the left anatomy and the right anatomy. As an example, the left-right opposite position of a label located on the left anatomy is the right anatomy.

The term “location for surgery” as used herein has the meaning a portion of the body on which a medical procedure is to be performed. A location for surgery may include the entirety of a body part or may only be a certain side or portion of a body part. Depending on the procedure to be performed, it is possible that two or more body parts or two or more portions of a body part may be considered a location for surgery.

The term “location not for surgery” as used herein has the meaning a portion or portions of the body which are not to be subjected to a medical procedure. A location not for surgery may include the entirety of a body part or may only be a certain side or portion of a body part. Depending on the procedure to be performed, it is possible that two or more body parts or two or more portions of a body part may be considered a location not for surgery.

The term “machine readable identifier” as used herein has the meaning any representation of data that may be scanned or read by a machine. As an example, a machine readable identifier may be a bar code, a QR Code, a magnetic stripe, or RFID tags.

The term “main sheet” as used herein has the meaning a primary surface for a label. The main sheet may be located between a separate bottom surface and top surface.

The term “medial” as used herein has the meaning toward the middle of the body or a body part. As an example, the medial portion of a knee is the inner portion of the knee.

The term “medial-lateral opposite” as used herein has the meaning the opposing side in terms of the medial anatomy and the lateral anatomy. As an example, the medial-lateral opposite position of a label located on the medial anatomy is on the lateral anatomy.

The term “medical data” as used herein has the meaning information that includes a patient's medical health history and which includes information pertaining to any current or future medical procedures or care to be provided to the patient.

The term “medical procedure” as used herein has the meaning a course of action followed by a doctor or a medical professional to provide care to a patient. A medical procedure may include a surgery or an operation, or any other form of medical care.

The term “negative indicia” as used herein has the meaning readable indicia that indicate that a certain body part or a portion of a certain body part is not to be subject to a medical procedure. As an example, affirmative indicia may include the words “no”, “no cut”, “no surgery”, “not correct”, “wrong”, “wrong site”, “negative”, or could be the color red, or could be a symbol such as a circle with a slash. Additionally, any other predetermined word, color, or symbol that indicates a site is not to be subject to a medical procedure may be used as negative indicia.

The term “readable indicia” as used herein has the meaning words, colors, bar codes, symbols, or any other visible form that conveys information. As an example, the information conveyed by readable indicia could be whether or not the body part labeled by the indicia is to be subject to a medical procedure. Readable indicia may also convey patient information, may provide a warning or a reminder, or may provide any other desired information.

The term “removable backing” as used herein has the meaning a temporary exterior layer that is able to be taken away from a label of a main sheet. As an example, the removable backing may be a covering that protects adhesive located on a label that is removed before the label is adhered to an object.

The term “right anatomy” as used herein has the meaning the right side of the body. As an example, a right arm and a right leg are part of the right anatomy.

The term “software” as used herein has the meaning a program or a series of operating instructions that may be performed by a computing device. The computing device may be a computer, a smartphone, a tablet, or any other computational machine.

The term “suitable for adhering to the skin” as used herein has the meaning that a substance has the property that the substance may be applied to the skin to allow adhesion of another object or material to the skin. The adhesive may be any composition that is safe to apply to the skin.

The term “tattoo indicia” as used herein has the meaning any readable indicia that are suitable for adhering directly to skin. Tattoo indicia may be a temporary ink or other kind of marking material that transfers onto a patient's skin. Tattoo indicia may include affirmative indicia, negative indicia, confirmation indicia, and/or identification indicia.

The term “top surface” as used herein has the meaning an exterior layer found on the upper most side of the main sheet and located on the side opposite the bottom surface.

While the invention has been illustrated and described in detail in the drawings and foregoing description, the same is to be considered as illustrative and not restrictive in character, it being understood that only the preferred embodiment has been shown and described and that all changes, equivalents, and modifications that come within the spirit of the inventions defined by following claims are desired to be protected. All publications, patents, and patent applications cited in this specification are herein incorporated by reference as if each individual publication, patent, or patent application were specifically and individually indicated to be incorporated by reference and set forth in its entirety herein.

1. A labeling system for use on a human surgical patient, comprising:
a label including a main sheet having a top surface and a bottom surface;
adhesive on said bottom surface and suitable for adhering to the skin of the patient;
removable backing covering said adhesive prior to use; and,
readable indicia on said main sheet and visible on or through said top surface,
wherein said readable indicia includes:
a) affirmative indicia indicating a location for surgery; and,
b) negative indicia indicating a location not for surgery.

2. The labeling system claim 1, comprising:
said affirmative indicia and said negative indicia being differentiated from each other with one corresponding to medial anatomy and the other corresponding to lateral anatomy.

3. The labeling system claim 1, comprising:
said affirmative indicia and said negative indicia being differentiated from each other with one corresponding to left anatomy and the other corresponding to right anatomy.

4. The labeling system of claim 1, comprising:
said affirmative indicia being selected from the group consisting of words or symbols including one or more of: “yes”, “cut”, “surgery”, “correct”, “right”, “affirmative”, the color green.

5. The labeling system of claim 1, comprising:
said negative indicia being selected from the group consisting of words or symbols including one or more of: “no”, “no cut”, “no surgery”, “not correct”, “wrong”, “wrong site”, “negative”, the color red.

6. The labeling system of claim 1, comprising:
said readable indicia includes identifying indicia on said main sheet and visible on or through said top surface, wherein said identifying indicia includes information corresponding to the patient's identity, the planned surgery for the patient, or both.

7. The labeling system of claim 6, wherein said readable indicia includes at least one machine readable identifier corresponding to the patient's identity, the planned surgery for the patient, or both.

8. The labeling system claim 1, wherein said readable indicia include at least one arrow.

9. The labeling system of claim 1, comprising:
said readable indicia includes confirmation indicia on said main sheet and visible on or through said top surface, wherein said readable indicia includes the term “confirm laterality”.

10. The labeling system claim 1, further comprising readable tattoo indicia on or in said adhesive, said tattoo indicia adapted to transfer at least some of corresponding readable indicia onto the skin of the patient.

11. The labeling system claim 6, further comprising readable tattoo indicia on or in said adhesive, said tattoo indicia adapted to transfer at least some of corresponding readable indicia onto the skin of the patient.

12. The labeling system claim 7, further comprising readable tattoo indicia on or in said adhesive, said tattoo indicia adapted to transfer at least some of corresponding readable indicia onto the skin of the patient.

13. A labeling system for use on a human surgical patient, comprising:
a main sheet having a top surface and a bottom surface;
adhesive on said bottom surface and suitable for adhering to the skin of the patient;
a removable backing covering said adhesive prior to use; and,
tattoo indicia on or in said adhesive, said tattoo indicia adapted to transfer at least some corresponding readable indicia onto the skin of the patient.

14. The labeling system of claim 13, wherein said readable indicia transferred onto the skin of the patient includes affirmative indicia indicating a location for surgery.

15. The labeling system of claim 13, wherein said readable indicia transferred onto the skin of the patient includes negative indicia indicating a location not for surgery.

16. The labeling system of claim 14, wherein said readable indicia transferred onto the skin of the patient includes negative indicia indicating a location not for surgery.

17. The labeling system of claim 16, wherein said affirmative indicia and said negative indicia are differentiated from each other with one corresponding to medial anatomy and the other corresponding to lateral anatomy.

18. The labeling system of claim 14, comprising:
said affirmative indicia being selected from the group consisting of words or symbols including one or more of: “yes”, “cut”, “surgery”, “correct”, “right”, “affirmative”, the color green.

19. The labeling system of claim 15, comprising:
said negative indicia being selected from the group consisting of words or symbols including one or more of: “no”, “no cut”, “no surgery”, “not correct”, “wrong”, “negative”, the color red.

20. A method for indicating on a human surgical patient, comprising the acts of:
(a) providing a label according to claim 1 by first providing label sheet that is at least partially unprinted;
(b) storing in a computer database information about the patient including: (i) patient identifying information including at least the patient's name; and, (ii) at least one prescribed surgery for the patient to receive;
(c) storing in a computer database separate database records about several various types of surgeries, including in such records: (i) surgery identifier; and, (ii) information to associate that surgery identifier with information to be printed on the surgery label;
(d) associating in a computer database said prescribed surgery for the patient to receive and said information to be printed on the surgery label; and,
(e) custom printing in said label sheet that is at least partially unprinted said information to be printed on the surgery label to create said label according to claim 1 which custom corresponds to said patient and said patient's prescribed surgery.

21. The method of claim 20, wherein said label sheet that is at least partially unprinted comprises at least two separate pre-cut-out portions on a common label backing; and wherein said information to be printed on at least a first of said pre-cut-out portions both: (a) is according to claim 1; and, (b) is templated from said computer database to print, during said act of printing, at a location on said label sheet on said first of said pre-cut-out portions.

22. The method of claim 20, wherein said act of printing includes printing patient identifying information, including in human readable form at least the patient's name, on said label sheet.

23. The method of claim 20 wherein said act of custom printing includes printing custom machine readable indicia that custom identifies said patient on said label according to claim 1.
